(19) United States
(12) Patent Application Publication
(10) Pub. No.: US 2025/0152149 A1
(43) Pub. Date: May 15, 2025

(54) BIOPSY NEEDLE HAVING THREE USAGE MODES

(71) Applicant: JINGFANG PRECISION MEDICAL DEVICE(SHENZHEN)CO., LTD., Shenzhen, Guangdong (CN)

(72) Inventors: Qingyuan Zeng, Shenzhen, Guangdong (CN); Weihua Zeng, Shenzhen, Guangdong (CN); Bo Feng, Shenzhen, Guangdong (CN)

(21) Appl. No.: 18/836,344

(22) PCT Filed: May 12, 2023
(86) PCT No.: PCT/CN2023/093918

§ 371 Date: Aug. 6, 2024

(30) Foreign Application Priority Data
Jul. 15, 2022 (CN) 202210838825.5

(51) Int. Cl. A61B 10/02

(52) U.S. Cl. CPC A61B 10/0233; A61B 2010/0208

(57) ABSTRACT

The present application relates to a biopsy needle having three usage modes, comprising an upper cover, a base, an inner needle assembly, an outer needle assembly, a loading assembly, a first rear emission assembly, a second rear emission assembly, a first side emission assembly and a second side emission assembly. The outer needle assembly and the inner needle assembly are loaded in sequence by the loading assembly, the inner needle assembly is emitted by the first rear emission assembly or the first side emission assembly, the outer needle assembly is emitted by the second rear emission assembly or the second side emission assembly, and the inner needle assembly can also be emitted by the second rear emission assembly or the second side emission assembly when it is not emitted, so that three usage modes are provided for users, and the users can select a rear emission or side emission operation.

### FIELD

The present application generally relates to medical devices, more particularly to a biopsy needle having three usage modes.

### BACKGROUND

In recent years, tumors have been lurking around human beings, and an incidence rate of tumors has gradually increased. Early detection and timely treatment can greatly prevent the progression of tumors, which requires ultrasound-guided biopsy of lesion cells or tissues for pathological analysis and diagnosis. A very important tool used for biopsy sampling is a biopsy needle. The biopsy needles are divided into manual biopsy needles, semi-automatic biopsy needles, and fully automatic biopsy needles. At present, existing fully automatic biopsy needles have a relatively single emission mode, which can only be triggered once or twice. Their emission operation manners are also limited. It is difficult for users to choose a most suitable emission mode and a most suitable operation manner according to different situations.

### SUMMARY

The technical problem to be solved by the present application is to provide a biopsy needle with three usage modes which is convenient for operations.

The technical solution adopted by the present application to solve the technical problem thereof is to provide a biopsy needle, comprising an upper cover, a base, an inner needle assembly, and an outer needle assembly, wherein the inner needle assembly comprises an inner needle holder and an inner needle fixedly installed on the inner needle holder, the outer needle assembly comprises an outer needle holder and an outer needle fixedly installed on the outer needle holder, the inner needle holder and the outer needle holder are respectively installed in the upper cover and the base that are combined with each other in a reciprocating sliding and elastic loading manner in a needle insertion direction, and the inner needle and the outer needle extend from front end in the needle insertion direction of the upper cover and the base, and the inner needle is inserted into the outer needle, and the biopsy needle further comprises a loading assembly, a first rear emission assembly, a second rear emission assembly, a first side emission assembly and a second side emission assembly, wherein:
the loading assembly is slidably set on the upper cover back and forth in a direction parallel to the needle insertion direction, and a first loading matching structure is provided for the loading assembly and the outer needle holder respectively, by which the outer needle holder is driven to be elastically loaded in place when the loading assembly slides backward, and a second loading matching structure is provided for the loading assembly and the inner needle holder respectively, by which a cooperation is achieved after the outer needle holder is loaded in place to drive the inner needle holder to be elastically loaded in place with the loading assembly sliding backward;
the first rear emission assembly and the second rear emission assembly are respectively installed at rear end in the needle insertion direction in the upper cover and the base combined with each other, and the first rear emission assembly has a first rear emission button which acts on the inner needle holder to release a loading state of the inner needle holder and emit the inner needle holder, and the second rear emission assembly has a second rear emission button which acts on the outer needle holder to release a loading state of the outer needle holder and emit the outer needle holder, and acts on the inner needle holder to release the loading state of the inner needle holder and emit the inner needle holder when the inner needle holder has not been emitted; and
the first side emission assembly and the second side emission assembly are respectively installed on one side in the needle insertion direction in the upper cover and the base combined with each other, and the first side emission assembly has a first side emission button which acts on the inner needle holder to release the loading state of the inner needle holder and emit the inner needle holder, and the second side emission assembly has a second side emission button which acts on the outer needle holder to release the loading state of the outer needle holder and emit the outer needle holder, and acts on the inner needle holder to release the loading state of the inner needle holder and emit the inner needle holder when the inner needle holder has not been emitted.

In the biopsy needle according to an embodiment of the present application, the loading assembly comprises a loading button, a loading seat, and a first reset spring, wherein the loading seat is slidably installed in a loading slot that extends parallel to the needle insertion direction on the upper cover, the loading button is fixedly connected to the loading seat and exposed outside the upper cover, and the first reset spring is set between the loading seat and the upper cover for resetting of the loading seat after it slides backward along the loading slot.

In the biopsy needle according to an embodiment of the present application, the first loading matching structure comprises a loading rod extending downwards from a front end of the loading seat and a loading protrusion protruding on the outer needle holder, and the loading seat slides backwards along the loading slot to make the loading rod contact the loading protrusion, thereby driving the outer needle holder to slide backwards and be loaded in place.

In the biopsy needle according to an embodiment of the present application, the second loading matching structure comprises two loading V-shaped grooves set on both sides of a rear end of the loading seat and two tongue pieces extending forward from the inner needle holder, and a connecting rod is connected between the two tongue pieces, and the outer needle holder further has an actuating rod that is inserted between the two tongue pieces of the inner needle holder to push the connecting rod upwards after the outer needle holder is loaded in place, to achieve a cooperation between the two tongue pieces and the two loading V-shaped grooves, and the loading seat slides backward along the loading slot to make the two loading V-shaped grooves contact against the two tongue pieces of the inner needle holder, thereby driving the inner needle holder to slide backward and be loaded in place.

In the biopsy needle according to an embodiment of the present application, the inner needle holder has a first elastic sheet at its one side in the needle insertion direction, which extends diagonally forward relative to the needle insertion direction, and an inner needle loading spring is set between the inner needle holder and the base, and when the inner needle holder slides backwards driven by the loading assembly till a front end of the first elastic sheet abuts to a corresponding first loading limiting surface in the base, the inner needle holder is loaded in place; and the outer needle holder has a second elastic sheet at its corresponding side in the needle insertion direction, which extends diagonally forward relative to the needle insertion direction, an outer needle loading spring is set between the outer needle holder and the base, and when the outer needle holder slides backwards driven by the loading assembly till a front end of the second elastic sheet abuts to a corresponding second loading limiting surface in the base, the outer needle holder is loaded in place.

In the biopsy needle according to an embodiment of the present application, both of the first rear emission button and the second rear emission button are installed on left and right sides at the rear end of the base in a compressible and elastic-reset manner, wherein the first rear emission button has a first rear emission member that extends towards the first elastic sheet of the inner needle holder, and when the first rear emission button is pressed, the first rear emission member pushes forward the first elastic sheet which abuts to the first loading limiting surface to make the first elastic sheet to detach from the first loading limiting surface and be released, thereby the inner needle holder and the inner needle are emitted under elastic force of the inner needle loading spring; and the second rear emission button has a second rear emission member that extends towards the second elastic sheet of the outer needle holder, and when the second rear emission button is pressed, the second rear emission member pushes forward the first elastic sheet of the inner needle holder that has not yet been emitted to make the first elastic sheet to detach from the first loading limiting surface and be released, thereby the inner needle holder and the inner needle are emitted under elastic force of the inner needle loading spring, and when the second rear emission button is continued to be pressed, the second rear emission member pushes forward the second elastic sheet of the outer needle holder that abuts to the second loading limiting surface to make the second elastic sheet to detach from the second loading limiting surface and be released, thereby the outer needle holder and the outer needle are emitted under elastic force of the outer needle loading spring.

In the biopsy needle according to an embodiment of the present application, both of the first side emission button and the second side emission button are slidably installed back and forth in an installation position formed on one side of the upper cover and the base combined with each other, and the first side emission button is fixedly connected to the first rear emission member of the first rear emission button, and the second side emission button is fixedly connected to the second rear emission member of the second rear emission button.

In the biopsy needle according to an embodiment of the present application, both of the first side emission button and the second side emission button are installed in a compressible and elastic-reset manner in an installation position formed on one side of the upper cover and the base combined with each other; and when the first side emission button is pressed, the first side emission button pushes inward the first elastic sheet which abuts to the first loading limiting surface to make the first elastic sheet to detach from the first loading limiting surface and be released, thereby the inner needle holder and the inner needle are emitted under elastic force of the inner needle loading spring; and when the second side emission button is pressed, the second side emission button pushes inward the first elastic sheet of the inner needle holder that has not yet been emitted to make the first elastic sheet to detach from the first loading limiting surface and be released, thereby the inner needle holder and the inner needle are emitted under elastic force of the inner needle loading spring, and when the second side emission button is continued to be pressed, the second side emission button pushes inward the second elastic sheet of the outer needle holder that abuts to the second loading limiting surface to make the second elastic sheet to detach from the second loading limiting surface and be released, thereby the outer needle holder and the outer needle are emitted under elastic force of the outer needle loading spring.

In the biopsy needle according to an embodiment of the present application, the inner needle is fixedly set at a lower end of the inner needle holder, the outer needle is fixedly set at a lower end of the outer needle holder, and the inner needle holder and the outer needle holder are both limited from top, bottom, left and right in an inner space provided by the upper cover and the base combined with each other, to only move forward and backward in the needle insertion direction.

In the biopsy needle according to an embodiment of the present application, a partition plate is set in the upper cover and the base combined with each other, for separating the first rear emission button and the second rear emission button.

Implementing the biopsy needle of the present application has the following beneficial effects: the biopsy needle according to the embodiment of the present application realize sequent loading of the outer needle assembly and the inner needle assembly by a single loading assembly, and provides a first rear emission assembly, a first side emission assembly, a second rear emission assembly, and a second side emission assembly for the inner needle assembly and the outer needle assembly separately, wherein the outer needle assembly is emitted by the second rear emission assembly or the second side emission assembly, and the inner needle assembly can also be emitted by the second rear emission assembly or the second side emission assembly when it has not been emitted, thereby three usage modes including a fully automatic emission mode, a delayed fully automatic emission mode, and a fully automatic zero range mode are provided for users to choose according to their actual needs, and it can facilitate users to flexibly choose rear emission or side emission operations according to actual situations.

### BRIEF DESCRIPTION OF THE DRAWINGS

The present application will be further described with reference to the accompanying drawings and embodiments, in which:

FIG. 1 is a schematic structural diagram of a biopsy needle with three usage modes according to a first embodiment of the present application;

FIG. 2 is a schematic structural diagram of an upper cover of the biopsy needle shown in FIG. 1;

FIG. 3 is a schematic structural diagram of the upper cover shown in FIG. 2 from another perspective;

FIG. 4 is a schematic structural diagram of a base of the biopsy needle shown in FIG. 1;

FIG. 5 is a schematic structural diagram of the base shown in FIG. 4 from another perspective;

FIG. 6 is a schematic structural diagram of a loading seat according to the first embodiment of the present application;

FIG. 7 is a schematic structural diagram of the loading seat shown in FIG. 6 from another perspective;

FIG. 8 is a schematic structural diagram of an inner needle holder according to the first embodiment of the present application;

FIG. 9 is a structural sectional view of the inner needle holder shown in FIG. 8;

FIG. 10 is a schematic structural diagram of an outer needle holder according to the first embodiment of the present application;

FIG. 11 is a structural sectional view of the outer needle holder shown in FIG. 10;

FIG. 12 is a partial structural sectional view of the inner needle holder in conjunction with the upper cover and the base according to the first embodiment of the present application;

FIG. 13 is a partial structural sectional view of the outer needle holder in conjunction with the upper cover and the base according to the first embodiment of the present application;

FIG. 14 is a structural sectional view of the biopsy needle according to the first embodiment of the present application, with both the inner needle assembly and the outer needle assembly in their initial states;

FIG. 15 is a structural sectional view of the biopsy needle according to the first embodiment of the present application, with the outer needle assembly in a loaded state;

FIG. 16 is a structural sectional view of the biopsy needle according to the first embodiment of the present application, with both the inner needle assembly and the outer needle assembly in their loaded states;

FIG. 17 is a schematic structural diagram of a first rear emission assembly according to the first embodiment of the present application;

FIG. 18 is a schematic structural diagram of a second rear emission assembly according to the first embodiment of the present application;

FIG. 19 is an internal structural diagram of the biopsy needle according to the first embodiment of the present application after the upper cover and the loading assembly thereon being removed;

FIG. 20 is a schematic structural diagram of a biopsy needle with three usage modes according to a second embodiment of the present application;

FIG. 21 is an internal structural diagram of the biopsy needle shown in FIG. 20 after the upper cover and the loading assembly thereon being removed.

### DETAILED DESCRIPTION

To explain objects, technical solutions and advantages of the present application more clearly, the present application will be further described with reference to the accompanying drawings and embodiments in the following. It should be understood that, the specific embodiments described here are only for explanation, but not for limitation to the present application. And, the embodiments and features in the embodiments of the present application can be combined with each other without conflict.

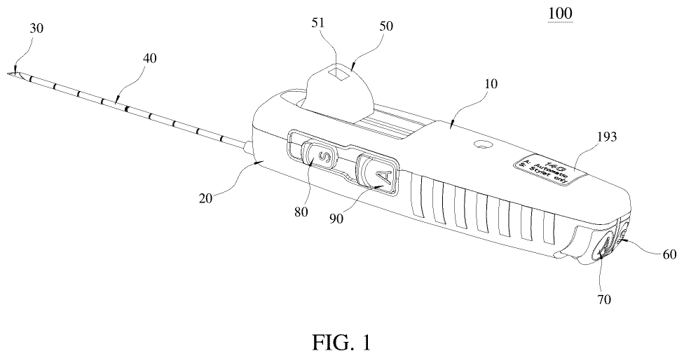

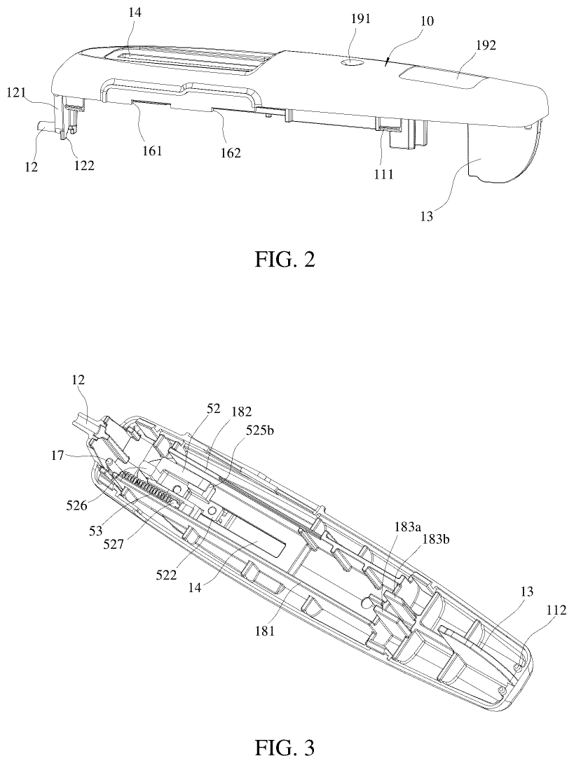

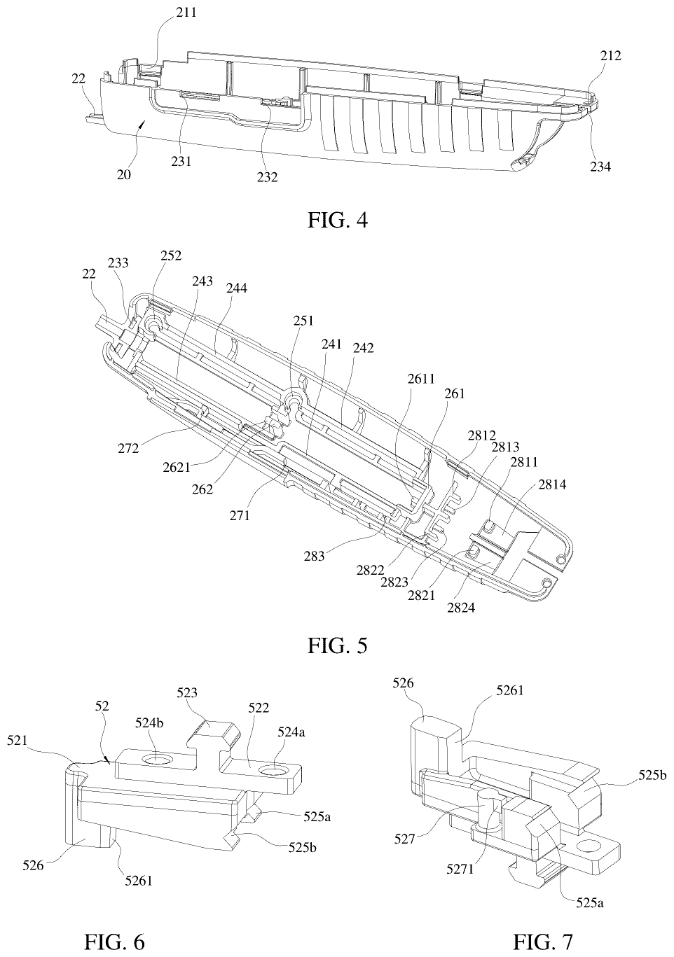

FIG. 1 shows a schematic structural diagram of a biopsy needle 100 with three usage modes according to a first embodiment of the present application. As shown in FIG. 1, the biopsy needle 100 with three usage modes comprises an upper cover 10, a base 20, an inner needle assembly 30, an outer needle assembly 40, a loading assembly 50, a first rear emission assembly 60, a second rear emission assembly 70, a first side emission assembly 80, and a second side emission assembly 90. The upper cover 10 and the base 20 are combined to enclose an internal space. The inner needle assembly 30 and the outer needle assembly 40 are respectively installed in the upper cover 10 and the base 20 in a reciprocating sliding and elastic loading manner in a needle insertion direction. An inner needle 31 of the inner needle assembly 30 and an outer needle 41 of the outer needle assembly 40 extend from front end in the needle insertion direction of the upper cover 10 and the base 20, and the inner needle 31 is inserted into the outer needle 41. The loading assembly 50 is slidably set on the upper cover 10 in a direction parallel to the needle insertion direction and is used to apply a backward moving force to the outer needle assembly 40 and the inner needle assembly 30 respectively in order to put the outer needle assembly 40 and the inner needle assembly 30 in a loading state. The first rear emission assembly 60 and the second rear emission assembly 70 are arranged left and right respectively at rear end in the needle insertion direction of the upper cover 10 and the base 20. The first side emission assembly 80 and the second side emission assembly 90 are arranged front and back respectively on left side of the upper cover 10 and the base 20. The first rear emission assembly 60 is used to act on the inner needle assembly 30 to release the loading state and emit the inner needle assembly 30. Similarly, the first side emission assembly 80 is also used to act on the inner needle assembly 30 to release the loading state and emit the inner needle assembly 30. The second rear emission assembly 70 is used to act on the outer needle assembly 40 to release the loading state and emit the outer needle assembly 40, and the second rear emission assembly 70 is also used to act on the inner needle assembly 30 to release the loading state and emit the inner needle assembly 30 when the inner needle assembly 30 is not emitted. Similarly, the second side emission assembly 90 is used to act on the outer needle assembly 40 to release the loading state and emit the outer needle assembly 40, and the second side emission assembly 90 is also used to act on the inner needle assembly 30 to release the loading state and emit the inner needle assembly 30 when the inner needle assembly 30 is not emitted. In the biopsy needle 100 according to the first embodiment of this application, three usage modes are achieved by a combination of the loading assembly 50, the first emission assembly 60, the second rear emission assembly 70, the first side emission assembly 80, and the second side emission assembly 90. This facilitates doctors to choose a most suitable emission mode according to different situations when using it, providing patients with a most timely and suitable biopsy plan. The following will provide a more detailed introduction in conjunction with FIGS. 2 to 19.

As shown in FIGS. 2 to 5, the upper cover 10 and the base 20 are combined with each other by matching buckling structures. The upper cover 10 is equipped with a plurality of buckles 111 along its inner circumference and two first positioning pillars 112 at the rear end. Correspondingly, the base 20 is equipped with a plurality of buckling grooves 211 along its inner circumference and two positioning holes 212 at the rear end. When the two first positioning pillars 112 of the upper cover 10 are aligned with and inserted into the two positioning holes 212 of the base 20 and the plurality of buckles 111 are engaged with the plurality of buckling grooves 211, the upper cover 10 is combined with the base 20. A first needle outlet fitting member 12 and a second needle outlet fitting member 22 are respectively set at the front end in the needle insertion direction of the top cover 10 and the base 20, and both of them surround to form a needle outlet close to a lower portion of the base 20 when the top cover 10 and the base 20 are combined, for the outer needle 41 and the inner needle 31 inserted through the outer needle 41 to be loaded back or be emitted through the needle outlet. Specifically as shown in FIG. 2 and FIG. 3, the first needle outlet fitting member 12 is located at a lower end of a support arm 121 that extends downwards from the front end of the upper cover 10, and two second positioning pillars 122 are set at the lower end of the support arm 121, which protrude downwards on both sides of the first needle outlet fitting member 12 respectively. Correspondingly, as shown in FIG. 5, two positioning grooves 233 are formed at the front end of the base 20 on both sides of the second needle outlet fitting member 22 respectively. When the upper cover 10 is combined with the base 20, the second positioning pillars 122 are embedded in the positioning grooves 233 to prevent deformation of the front end of the biopsy needle 100 after being assembled. Moreover, the needle outlet formed by the first needle outlet fitting member 12 and the second needle outlet fitting member 22 are arranged close to the lower end of the base 20, making it easier for the inner needle 31 and the outer needle 41 to be used in conjunction with auxiliary devices such as ultrasound probes. In order to make axis of the inner needle 31 and the outer needle 41 close to the base 20 as much as possible, a direction of elastic loading emission force will inevitably be offset from the axis of the inner needle and the outer needle and not coaxial, thus it is necessary to jointly provide a limiting structure in the upper cover 10 and the base 20 that can make the inner needle holder 32 and the outer needle holder 42 slide back and forth without causing any deviation, to ensure that the inner needle 31 and the outer needle 41 do not produce any uneven deviation during needle emission. A partition plate 13 is equipped at the rear end in the needle insertion direction of the upper cover 10, which extends towards the base 20, and a groove 234 for insertion of the partition plate 13 is formed at the rear end of the base 20. When the upper cover 10 is combined with the base 20, the partition plate 13 is inserted into the groove 234 to divide open space at the rear end of the base 20 into two portions, which are used for installing the first rear emission assembly 60 and the second rear emission assembly 70, respectively. On a left side of the upper cover 10, a first installation groove 161 and a second installation groove 162 are arranged forward and backward along the needle insertion direction, and correspondingly on a left side of the base 20, a third installation groove 231 and a fourth installation groove 232 are arranged forward and backward along the needle insertion direction. When the upper cover 10 is combined with the base 20, the first installation groove 161 and the third installation groove 231 jointly surround to form an installation position for installing the first side emission assembly 80, and the second installation groove 162 and the fourth installation groove 232 jointly surround to form an installation position for installing the second side emission assembly 90.

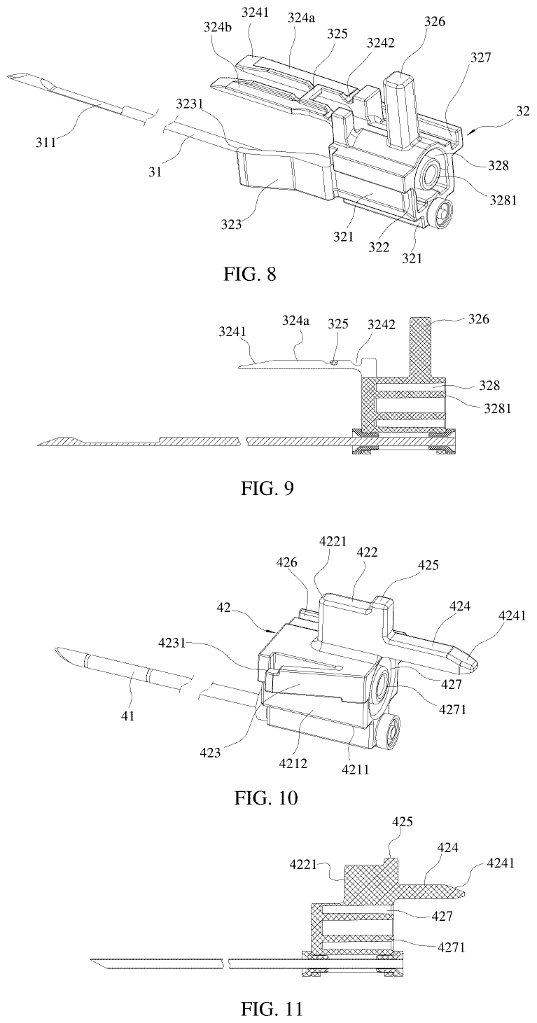

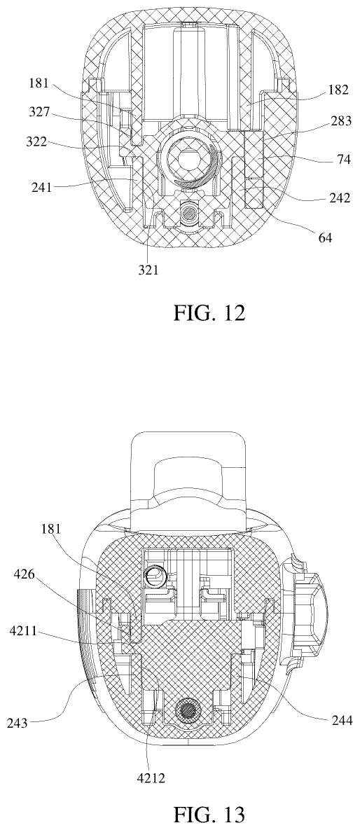

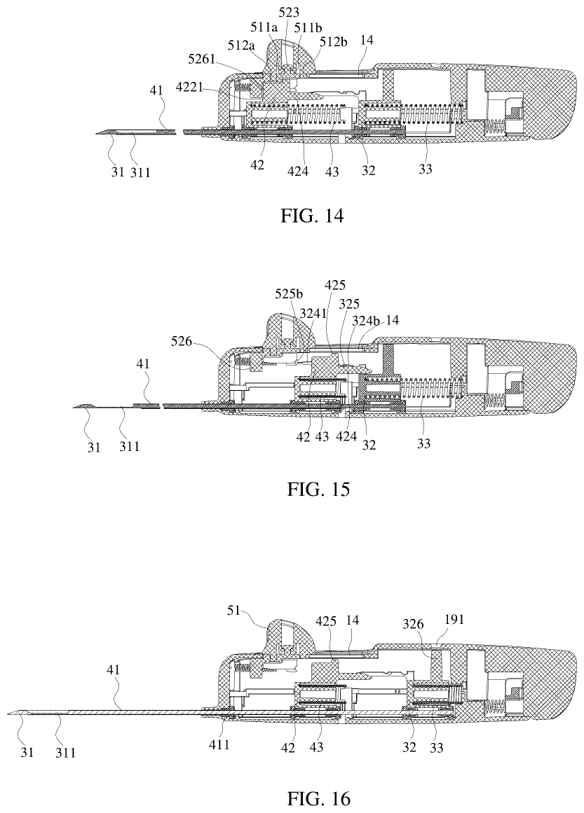

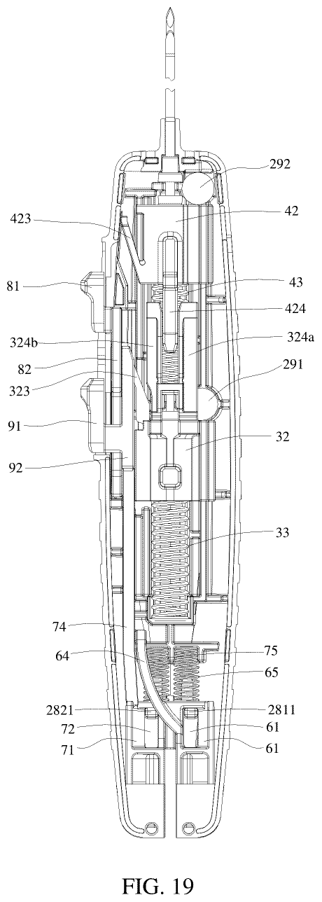

Further referring to FIG. 8 and FIG. 9, combined with FIG. 14 and FIG. 19, the inner needle assembly 30 comprises an inner needle 31, an inner needle holder 32, and an inner needle loading spring 33. Specifically as shown in FIG. 8 and FIG. 9, the inner needle 31 is a solid needle rod with a sampling groove 311 at its front end, and its rear end is fixed at a lower end of the inner needle holder 32 in the needle insertion direction (such as fixed by adhesive or integrated injection molding), so that the inner needle 31 can move together with the inner needle holder 32. A first identification pillar 326 that extends upwards is equipped at an upper end in the needle insertion direction of the inner needle holder 32, and two tongue pieces 324a and 324b that extend horizontally forward at a certain distance apart are equipped on the front side of the upper end of the inner needle holder 32. A connecting rod 325 is connected between the two tongue pieces 324a and 324b and loading slopes 3241 are formed at the front end of the two tongue pieces 324a and 324b respectively, and channels 3242 are formed on upper surface of the rear ends of the two tongue pieces 324a and 324b, respectively. The inner needle holder 32 has a first elastic sheet 323 at its left side in the needle insertion direction, which extends diagonally forward relative to the needle insertion direction, and a first contact surface 3231 is formed at a front end of the first elastic sheet 323. The inner needle holder 32 has a first guiding groove 327 on its right side in the needle insertion direction, and the first guiding groove 327 extends along the needle insertion direction. The inner needle holder 32 also has a first spring mounting hole 328 at its rear end in the needle insertion direction, and a first spring guiding pillar 3281 is set in the first spring mounting hole 328 for installing the inner needle loading spring 33. As shown in combination with FIGS. 3, 5, 12, 14, and 19, the inner needle holder 32 is slidably installed at the rear of the internal space provided by the upper cover 10 and the base 20 relative to the outer needle holder 42. On the left and right sides in the base 20, there are a first limiting protrusion 241 and a second limiting protrusion 242 that extend upwards corresponding to the inner needle holder 32. On the left and right sides in the upper cover 10, there are a guiding protrusion 181 and a limiting rib 182 that extend downwards corresponding to the inner needle holder 32. The inner needle holder 32 has two left and right side surfaces 321 which are opposite to and contact with inner surfaces of the first limiting protrusion 241 and the second limiting protrusions 242 for left and right limitation, and the inner needle holder 32 also has bottom surfaces 322 on its both sides for supporting on top of the first limiting protrusion 241 and the second limiting protrusion 242 for downward limitation. Furthermore, the guiding protrusion 181 in the upper cover 10 slidably inserts into the first guiding groove 327 of the inner needle holder 32, and the limiting rib 182 presses against the top surface of the inner needle holder 32. These not only provide guiding effect in the needle insertion direction, but also provide upward limitation for the inner needle holder 32 (see FIG. 12). In this way, the inner needle holder 32 is limited in the internal space of the upper cover 10 and the base 20 and can only move forward and backward in the needle insertion direction without any axial deviation. As shown in FIG. 14, a front end of the inner needle loading spring 33 is pressed against the first spring mounting hole 328 of the inner needle holder 32, and a rear end of the inner needle loading spring 33 is fitted into a first spring mounting seat 261 in the base 20 and supported by a first spring support platform 2611 (as shown in FIG. 5). If a force is applied to move the inner needle holder 32 backwards, the inner needle loading spring 33 will be compressed. When the inner needle holder 32 is moved backwards and the first contact surface 3231 at the front end of the first elastic sheet 323 is pressed against a first loading limiting surface 271 on the inner wall of the base 20 (as shown in FIG. 5), the inner needle holder 32 is loaded in place, and the elastic force of the inner needle loading spring 33 causes the first contact surface 3231 of the first elastic sheet 323 to tightly press against the first loading limiting surface 271 on the inner wall of the base 20.

Further referring to FIG. 10 and FIG. 11 combined with FIG. 14 and FIG. 19, the outer needle assembly 40 comprises an outer needle 41, an outer needle holder 42, and an outer needle loading spring 43. Specifically, as shown in FIG. 10 and FIG. 11, the outer needle 41 is a hollow needle tube, and its rear end is fixed at a lower end in the needle insertion direction of the outer needle holder 42 to move together with the outer needle holder 42. The outer needle holder 42 has a loading protrusion 422 at its upper end in the needle insertion direction, with a loading matching surface 4221 being formed in the front end of the loading protrusion 422 and an actuating rod 424 extending horizontally from the rear end of the loading protrusion 422. The actuating rod 424 has an actuating inclined surface 4241 at its rear end. There is also a second identification pillar 425 extending upwards on the loading protrusion 422. The outer needle holder 42 has a second elastic sheet 423 at its left side in the needle insertion direction, which extends diagonally forward relative to the needle insertion direction, and a second contact surface 4231 is formed at a front end of the second elastic sheet 423. The outer needle holder 42 has a second guiding groove 426 on its right side in the needle insertion direction, and the second guiding groove 426 extends along the needle insertion direction. The outer needle holder 42 also has a second spring mounting hole 427 at its rear end in the needle insertion direction, and a second spring guiding pillar 4271 is set in the second spring mounting hole 427 for installing the outer needle loading spring 43. As shown in combination with FIGS. 3, 5, 13, 14, and 19, the outer needle holder 42 is slidably installed in the front of the internal space provided by the upper cover 10 and the base 20 relative to the inner needle holder 32. A front end of the outer needle 41 passes through the needle outlet formed by the upper cover 10 and the base 20 with a shaft sleeve 411 to ensure coaxiality, and the inner needle 31 is inserted into the outer needle 41 from the rear end of the outer needle 41. In an initial state, the sampling groove 311 at the front end of the inner needle 31 is stored in the front end of the outer needle 41. On the left and right sides in the base 20, there are a third limiting protrusion 243 and a fourth limit protrusion 244 that extend upwards corresponding to the outer needle holder 42. The guiding protrusions 181 and the limiting rib 182 set in the upper cover 10 also extend along the needle insertion direction to a position corresponding to the outer needle holder 42. The outer needle holder 42 has two left and right side surfaces 4212 that are opposite to and contact with inner surfaces of the third limiting protrusion 243 and the fourth limiting protrusion 244 for left and right limitation, and the outer needle holder 42 also has bottom surfaces 4211 on its both sides for supporting on top of the third limiting protrusion 243 and the fourth limiting protrusion 244 for downward limitation. Furthermore, the guiding protrusion 181 in the upper cover 10 slidably inserts into the second guiding groove 426 of the outer needle holder 42, and the limiting rib 182 presses against the top surface of the inner needle holder 32. These not only provide guiding effect in the needle insertion direction, but also provide upward limitation for the outer needle holder 42. In this way, the outer needle holder 42 is limited in the internal space of the upper cover 10 and the base 20 and can only move forward and backward in the needle insertion direction without any axial deviation. As shown in FIG. 14, a front end of the outer needle loading spring 43 is pressed against the second spring mounting hole 427 of the outer needle holder 42, and a rear end of the outer needle loading spring 43 is fitted onto a second spring mounting pillar 2621 of a second spring mounting seat 262 set in the base 20. If a force is applied to move the outer needle holder 42 backwards, the outer needle loading spring 43 will be compressed. When the outer needle holder 42 is moved backwards and the second contact surface 4231 at the front end of the second elastic sheet 423 is pressed against a second loading limiting surface 272 on the inner wall of the base 20 (as shown in FIG. 5), the outer needle holder 42 is loaded in place, and the elastic force of the outer needle loading spring 43 causes the second contact surface 4231 of the second elastic sheet 423 to tightly press against the second loading limiting surface 272 on the inner wall of the base 20.

Further referring to FIGS. 1, 6, and 7, combined with FIGS. 14 to 16, the loading assembly 50 comprises a loading button 51, a loading seat 52, and a first reset spring 53. A loading slot 14 extending parallel to the needle insertion direction is formed at the front of the upper cover 10, and the loading seat 52 is slidably installed in the loading slot 14, and the loading button 51 is fixedly connected to the loading seat 52 and exposed outside the upper cover 10. A backward force applied to the loading button 51 can drive the loading seat 52 to move backward along the loading slot 14, thereby acting on the outer needle holder 42 or the inner needle holder 32 to load the outer needle assembly 40 or the inner needle assembly 30 in place. The elastic force of the first reset spring 53 causes the loading seat 52 and the loading button 51 to reset to their initial positions after the backward force is released. Specifically, as shown in FIG. 6 and FIG. 7, the loading seat 52 has a loading slider 522 on its top surface 521, and a T-shaped protrusion 523 is set in the middle of the loading slider 522. A first limiting hole 524a and a second limiting hole 524b are formed on both sides of the loading slider 522 with respect to the T-shaped protrusion 523. On both sides of the rear end in the needle insertion direction of the loading seat 52, there are two loading V-shaped grooves 525a and 525b that are matched with the two tongue pieces 324a and 324b of the inner needle holder 32, and a loading rod 526 that is matched with the loading protrusion 422 of the outer needle holder 42 extends downwards at the front end in the needle insertion direction of the loading seat 52. Further as shown in FIG. 14, the loading seat 52 is slidably installed in the loading slot 14 of the upper cover 10 by means of the loading slider 522, wherein, the top surface 521 of the loading seat 52 is in contact with an inner surface of the upper cover 10 for upward limitation, two elastic buckles 511a and 511b in the loading button 51 are engaged with the T-shaped protrusion 523 for upward and downward limitation, and a first positioning pillar 512a and a second positioning pillar 512b of the loading button 51 are inserted into the first limiting hole 524a and the second limiting hole 524b on the loading slider 522 respectively, thereby the loading button 51 is fixedly installed on the loading seat 52 and moving the loading button 51 can drive the loading seat 52 to move forward and backward along the loading slot 14. Further as shown in FIG. 7 and FIG. 3, there is a third spring mounting pillar 527 at the bottom of the loading seat 52, and a fourth spring mounting pillar 17 is located in the upper cover 10 in front of the loading seat 52. Two ends of the first reset spring 53 are respectively connected to the third spring mounting pillar 527 and the fourth spring mounting pillar 17. The loading seat 52 will move backward along the loading slot 14 to stretch the first reset spring 53 when a backward force is applied, and after the backward force is released, the first reset spring 53 will drive the loading seat 52 move forward to reset. As shown in FIG. 7, there is also a stopper 5271 on the outer circumference of the third spring mounting pillar 572 to prevent the first reset spring 53 from escape.

A specific process of loading the inner needle assembly 30 and the outer needle assembly 40 by the loading assembly 50 will be explained in detail in conjunction with FIGS. 14 to 16. Firstly, as shown in FIG. 14, the loading assembly 50, the inner needle assembly 30, and the outer needle assembly 40 are all in their initial states, wherein, the sampling groove 311 of the inner needle 31 is stored in the front end of the outer needle 41. At this time, applying a backward force to the loading button 51 will drive the loading seat 52 to move backward along the loading slot 14. The loading surface 5261 of the loading rod 526, which extends downward at the front end of the loading seat 52, will first contact the loading matching surface 4221 of the loading protrusion 422 of the outer needle seat 42, to drive the outer needle holder 42 and the outer needle 41 to move backward together, with the actuating rod 424 at the rear end of the outer needle holder 42 being inserted into a space between the two tongue pieces 324a and 324b of the inner needle holder 32. When the outer needle holder 42 is moved backwards till the second contact surface 4231 at the front end of the second elastic sheet 423 abuts against the second loading limiting surface 272 on the inner wall of the base 20, the outer needle holder 42 is loaded in place. At this time, as shown in FIG. 15, the backward force on the loading button 51 is released, and the loading button 51 and the loading seat 52 are reset to the initial position under the action of the first reset spring 53. The outer needle loading spring 43 is compressed, and the outer needle 41 moves backward and retracts with the outer needle holder 42, the sampling groove 311 of the inner needle 31 is exposed. Moreover, at this time, the actuating rod 424 of the outer needle holder 42 contacts the connecting rod 325 between the two tongue pieces 324a and 324b of the inner needle holder 32, and the actuating rod 424 gradually pushes the connecting rod 325 upwards through the actuating inclined surface 4241 at its rear end, thereby driving the two tongue pieces 324a and 324b connected to the connecting rod 325 to deform and be pushed upwards around the channels 3242, causing the two tongue pieces 324a and 324b to be in a tilted state as shown in FIG. 15. In addition, after the outer needle assembly 40 is loaded in place, as shown in FIG. 15, the second identification pillar 425 on the outer needle holder 42 is exposed from the loading slot 14 for the user to observe and confirm the state. Then, a backward force is applied to the loading button 51 again to drive the loading seat 52 to move backward along the loading slot 14. As the outer needle holder 42 has been moved backwards and loaded in place, the loading rod 526 at the front end of the loading seat 52 no longer functions. The loading V-shaped grooves 525a and 525b at the rear end of the loading seat 52 will respectively collide with the two upward sloping tongue pieces 324a and 324b at the front end of the inner needle holder 32, and then drive the inner needle holder 32 and the inner needle 31 to move backward together. When the inner needle holder 32 is moved backwards till the first contact surface 3231 at the front end of the first elastic sheet 323 contacts the first loading limiting surface 271 on the inner wall of the base 20, the inner needle holder 32 is loaded in place. At this time, as shown in FIG. 16, the backward force on the loading button 51 is released, and the loading assembly 50 is reset to the initial position under the action of the first reset spring 53. The inner needle loading spring 33 is compressed, and the inner needle 31 moves backward and retracts along with the inner needle holder 32. The sampling groove 311 of the inner needle 31 is again stored in the front end of the outer needle 41. Moreover, when the inner needle holder 32 is loaded into place, as shown in FIG. 16, the first identification pillar 326 on the inner needle holder 32 is precisely exposed from an observation window 191 opened on the upper cover 10 for the user to observe and confirm the state.

As described above, in the biopsy needle 100 according to the first embodiment of the present application, elastic loading of the outer needle assembly 40 is achieved by a first loading matching structure composed of the loading rod 526 on the loading seat 52 and the loading protrusion 422 on the outer needle holder 42 between the loading assembly 50 and the outer needle assembly 40, and elastic loading of the inner needle assembly 30 is achieved by a second loading matching structure composed of the loading V-shaped grooves 525a and 525b on the loading seat 52 and the tongue pieces 324a and 324b on the inner needle holder 32 between the loading assembly 50 and the inner needle assembly 30, and only after the outer needle assembly 40 is loaded in place, the second loading matching structure between the loading assembly 50 and the inner needle assembly 30 cab achieve cooperation. Therefore, the biopsy needle 100 according to the first embodiment of the present application not only achieves loading of both the outer needle assembly 40 and the inner needle assembly 30 by means of a single loading assembly 50, but also achieves a sequential loading of the outer needle assembly 40 and the inner needle assembly 30 by means of a single loading assembly 50. In addition, according to different embodiments of the present application, the first loading matching structure between the loading assembly 50 and the outer needle assembly 40, as well as the second loading matching structure between the loading assembly 50 and the inner needle assembly 30, are not limited to the specific structures shown in the first embodiment. Other suitable matching structures can also be used, as long as it is possible to load the outer needle assembly 40 and the inner needle assembly 30 sequentially through a single loading assembly 50.

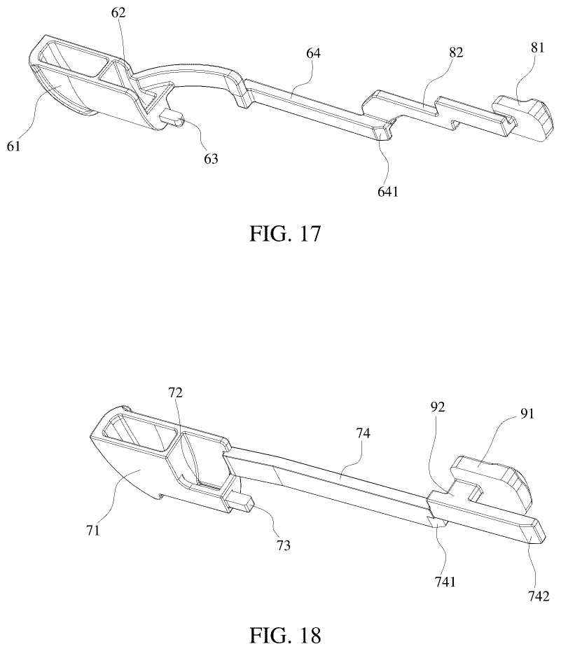

Furthermore, as shown in FIG. 17 and FIG. 19, the first rear emission assembly 60 comprises a first rear emission button 61 and a second reset spring 65. The first rear emission button 61 is installed in a compressible manner on left side of the open space at the rear end of the base 20 and can be reset by the second reset spring 65. As shown in FIG. 17, a first pressing channel 62 is formed at the bottom of the first rear emission button 61, a fifth spring mounting pillar 63 and a first rear emission member 64 are respectively set at the front end of the first rear emission button 61, and the first rear emission member 64 is preferably a rod-shaped component extending to the left front, and a first rear emission surface 641 is formed at its front end. The first side emission assembly 80 comprises a first side emission button 81 and a first emission connecting rod 82, and the first side emission assembly 80 is fixedly connected to the front end of the first rear emission member 64 through the first emission connecting rod 82, thereby being integrated with the first rear emission assembly 60. Further referring to FIG. 19 and FIG. 5, the first rear emission button 61 is installed in the base 20. A first support surface 2814 is formed on the right side of the bottom of the base 20, and a first bump 2811 is set on the first support surface 2814 and inserted into the first pressing channel 62 of the first rear emission button 61, so that a bottom surface of the first rear emission button 61 is supported on the first support surface 2814 of the base 20, and the first rear emission button 61 can slide forward and backward through a cooperation of the first bump 2811 and the first pressing channel 62. At the same time, there is also a guiding channel 283 (as shown in FIG. 5) in the base 20 for the first rear emission member 64 of the first rear emission button 61 being placed therein and being guided to move forward. One end of the second reset spring 65 is mounted on the fifth spring mounting pillar 63 against the first rear emission button 61, and the other end of the second reset spring 65 is mounted on a sixth spring mounting pillar 2813 of a third spring mounting seat 2812 set in the base 20 (see FIG. 5). In this way, applying a force of pressing the first rear emission button 61 forward can cause the first rear emission button 61 to move forward along the first pressing channel 62, thereby the a rear emission surface 641 of the first rear emission member 64 pushes forward the first elastic sheet 323 that abuts against the first loading limiting surface 271, causing the first elastic sheet 323 to detach from the first loading limiting surface 271 and the limitation is released, thus the inner needle holder 32 and the inner needle 31 are emitted under the elastic force of the inner needle loading spring 33. Releasing the force applied to the first rear emission button 61, the first rear emission button 61 can be reset under the elastic force of the second reset spring 65. Further as shown in FIG. 19, the first side emission button 81 of the first side emission assembly 80 is just slidably installed in an installation position formed by a combination of the first installation groove 161 of the upper cover 10 and the third installation groove 231 of the base 20 when the first rear emission assembly 60 is installed in place. Applying a force of pushing the first side emission button 81 forward can also drive the first rear emission button 61 to move forward along the first pressing channel 62, thereby the loading of the inner needle assembly 30 is released by the first rear emission member 64. When the force applied to the first side emission button 81 is released, the first side emission button 81 can be reset under the elastic force of the second reset spring 65.

Further referring to FIG. 18 and FIG. 19, the second rear emission assembly 70 comprises a second rear emission button 71 and a third reset spring 75. The second rear emission button 71 is installed in a compressible manner on right side of the open space at the rear end of the base 20 and can be reset by the third reset spring 75. As shown in FIG. 18, a second pressing channel 72 is formed at the bottom of the second rear emission button 71, a seventh spring mounting pillar 73 and a second rear emission member 74 are respectively set at the front end of the second rear emission button 71, and the second rear emission member 74 is preferably a rod-shaped component extending to the left front, and a second rear emission surface 741 is formed at its front end. The second rear emission member 74 also has a third rear emission surface 742 at a corresponding position behind the second rear emission surface 741. The second side emission assembly 90 comprises a second side emission button 91 and a second emission connecting rod 92, and the second side emission assembly 90 is fixedly connected to the front end of the second rear emission member 74 through the second emission connecting rod 92, thereby being integrated with the second rear emission assembly 70. Further referring to FIG. 19 and FIG. 5, the second rear emission button 71 is installed in the base 20. A second support surface 2824 is formed on the left side of the bottom of the base 20, and a second bump 2821 is set on the second support surface 2824 and inserted into the second pressing channel 72 of the second rear emission button 71, so that a bottom surface of the second rear emission button 71 is supported on the second support surface 2824 of the base 20 and can slide forward and backward through a cooperation of the second bump 2821 and the second pressing channel 72. At the same time, the second rear emission member 74 of the second rear emission button 71 is stacked above the first rear emission member 64 in the guiding channel 283 in the base 20 and is guided by the guiding channel 283 for forward movement. One end of the third reset spring 75 is mounted on the seventh spring mounting pillar 73 against the second rear emission button 71, while the other end of the third reset spring 75 is mounted on an eighth spring mounting pillar 2823 of a fourth spring mounting seat 2822 set in the base 20 (see FIG. 5). In this way, applying a force of pressing the second rear emission button 71 forward can cause the second rear emission button 71 to move forward along the second pressing channel 72, thereby the third rear emission surface 742 of the second rear emission member 74 pushes forward the first elastic sheet 323 that abuts against the first loading limiting surface 271, causing the first elastic sheet 323 to detach from the first loading limiting surface 271 and the limitation is released, thus the inner needle holder 32 and the inner needle 31 are emitted under the elastic force of the inner needle loading spring 33, and then the second rear emission surface 741 of the second rear emission member 74 pushes forward the second elastic sheet 423 that abuts against the second loading limiting surface 272, causing the second elastic sheet 423 to detach from the second loading limiting surface 272 and the limitation is released, thus the outer needle holder 42 and the outer needle 41 are emitted under the elastic force of the outer needle loading spring 43. To ensure successful sampling and cutting, there is a slight travel difference between the second rear emission surface 741 of the second rear emission member 74 pushing against the second elastic sheet 423 and the third rear emission surface 742 of the second rear emission member 74 pushing against the first elastic sheet 323. That is, the third rear emission surface 742 will first contact the first elastic sheet 323 to emit the inner needle holder 32 and the inner needle 31, and then the second rear emission surface 741 will contact the second elastic sheet 423 to emit the outer needle holder 42 and the outer needle 41. Releasing the force applied to the second rear emission button 71, the second rear emission button 71 can be reset under the elastic force of the third reset spring 75. Further as shown in FIG. 19, the second side emission button 91 of the second side emission assembly 90 is just slidably installed in an installation position formed by a combination of the second installation groove 162 of the upper cover 10 and the fourth installation groove 232 of the base 20 when the second rear emission assembly 70 is installed in place. Applying a force of pushing the second side emission button 91 forward can also drive the second rear emission button 71 to move forward along the second pressing channel 72, thereby the loading of the outer needle assembly 40 is released by the second rear emission member 74 and the outer needle 41 is emitted, and if the inner needle assembly 30 has not been emitted, the loading of the inner needle assembly 30 can also be released and the inner needle 31 can be emitted. Releasing the force applied to the second side emission button 91, the second side emission button 91 can be reset under the elastic force of the third reset spring 75. Furthermore, as shown in FIG. 3, a first limiting station 183a and a second limiting station 183b extend downwards in the upper cover 10, facing the second reset spring 65 and the third reset spring 75 respectively. When the upper cover 10 is combined with the base 20, the first limiting station 183a and the second limiting station 183b just block the second reset spring 65 and the third reset spring 75 from above, preventing them from escape and improving the smoothness of pressing.

As mentioned above, in the biopsy needle 100 according to the first embodiment of the present application, the first rear emission assembly 60 and the second rear emission assembly 70 are separately provided for the inner needle assembly 30 and the outer needle assembly 40, respectively, and the loading state of the inner needle assembly 30 is released and the inner needle assembly 30 is emitted by the first rear emission assembly 60 acting on the inner needle assembly 30, and the loading state of the outer needle assembly 40 is released and the outer needle assembly 40 is emitted by the second rear emission assembly 70 acting on the outer needle assembly 40, and in the case where the inner needle assembly 30 has not been emitted, the loading state of the inner needle assembly 30 can also be released and the inner needle assembly 30 can also be emitted by the second rear emission assembly 70 acting on the inner needle assembly 30. In addition, in the biopsy needle 100 according to the first embodiment of the present application, the first side emission assembly 80 and the second side emission assembly 90 are separately provided for the inner needle assembly 30 and the outer needle assembly 40, respectively, and the first side emission assembly 80 is linked to the first rear emission assembly 60, and the second side emission assembly 90 is linked to the second rear emission assembly 70. The first side emission assembly 80 acts on the inner needle assembly 30 to release the loading state of the inner needle assembly 30 and emit it. The second side emission assembly 90 acts on the outer needle assembly 40 to release the loading state of the outer needle assembly 40 and emit it. In the case where the inner needle assembly 30 has not yet been emitted, the inner needle assembly 30 can also be released and emitted by the second side emission assembly 90 acting on the inner needle assembly 30.

Furthermore, according to different embodiments of the present application, linkage method between the first rear emission member 64 of the first rear emission assembly 60, the second rear emission member 74 of the second rear emission assembly 70, the first side emission assembly 80 and the first rear emission assembly 60, and linkage method between the second side emission assembly 90 and the second rear emission assembly 70, are not limited to the specific structures shown in the first embodiment, and various other suitable structural forms can be adopted as long as the loading of the inner needle assembly 30 and the outer needle assembly 40 can be released. According to different embodiments of the present application, the second reset spring 65 and the third reset spring 75 can also be replaced by other elastic components, such as elastic strips or elastic sheets provided by the first rear emission button 61 and the second rear emission button 71, or other suitable elastic reset structures can be used for the first rear emission button 61 and the second rear emission button 71, as long as their respective pressing and resetting can be achieved.

The usage of the biopsy needle 100 with three usage modes according to the first embodiment of the present application is as follows:

### 1. Loading

1.1 As shown in FIG. 14 and FIG. 15, a backward force is applied to the loading button 51 to drive the loading seat 52 to move backward along the loading slot 14, thereby driving the outer needle holder 42 and the outer needle 41 to move backward together. When the second contact surface 4231 at the front end of the second elastic sheet 423 of the outer needle holder 42 contacts the second loading limiting surface 272 on the inner wall of the base 20, the outer needle holder 42 is loaded in place. At the same time, the actuating rod 424 of the outer needle holder 42 pushes the two tongue pieces 324a and 324b of the inner needle holder 32 upwards, causing the two tongue pieces 324a and 324b to be in an inclined state as shown in FIG. 15. Then the backward force on the loading button 51 is released, and the loading button 51 and the loading seat 52 reset to their initial positions under the action of the first reset spring 53.

1.2 As shown in FIG. 15 and FIG. 16, a backward force is again applied to the loading button 51 to drive the loading seat 52 to move backward along the loading slot 14, thereby driving the inner needle holder 32 and the inner needle 31 to move backward together. When the first contact surface 3231 at the front end of the first elastic sheet 323 of the inner needle holder 32 contacts the first loading limiting surface 271 on the inner wall of the base 20, the inner needle holder 32 is loaded in place. Then the backward force on the loading button 51 is released, and the loading button 51 and the loading seat 52 reset to their initial positions under the action of the first reset spring 53.

### 2. Emission

2.1 As shown in FIG. 19, pressure can be optionally applied to the first rear emission button 61 or the first side emission button 81, and then the first rear emission member 64 acts on the first elastic sheet 323 of the inner needle holder 32, causing it to detach from the first loading limiting surface 271 and the limitation is released. The inner needle holder 32 and the inner needle 31 are then emitted forward under the elastic force of the inner needle loading spring 33. Preferably, as shown in FIG. 5 and FIG. 19, a first installation hole 251 is provided in the base 20 at a front end of forward emission stroke of the inner needle holder 32, and a first buffer pad 291 is set inside the first installation hole 251, for buffering impact force of the inner needle holder 32 and reducing working noise of the biopsy needle 100.

2.2 As shown in FIG. 19, pressure can be optionally applied to the second rear emission button 71 or the second side emission button 91, and then the second rear emission surface 741 of the second rear emission member 74 acts on the second elastic sheet 423 of the outer needle holder 42, causing it to detach from the second loading limiting surface 272 and the limitation is released. The outer needle holder 42 and the outer needle 41 are then emitted forward under the elastic force of the outer needle loading spring 43. Preferably, as shown in FIG. 5 and FIG. 19, a second installation hole 252 is provided in the base 20 at a front end of forward emission stroke of the outer needle holder 42, and a second buffer pad 292 is set inside the second installation hole 252, for buffering impact force of the outer needle holder 42 and reducing working noise of the biopsy needle 100.

2.3 As shown in FIG. 19, pressure can be optionally applied to the second rear emission button 71 or the second side emission button 91, and the third rear emission surface 742 of the second rear emission member 74 first acts on the first elastic sheet 323 of the inner needle holder 32, causing the inner needle assembly 30 to be emitted forward, and then the second rear emission surface 741 of the second rear emission member 74 acts on the second elastic sheet 423 of the outer needle holder 42, causing the outer needle assembly 40 to be emitted forward.

### 3. Usage Mode

### 3.1 Fully Automatic Emission Mode (Automatic)

Firstly, the above loading steps 1.1 and 1.2 are performed, then the biopsy needle 100 is controlled at a safe distance from a lesion, and then the above emission step 2.3 is performed to sequentially emit the inner needle assembly 30 and the outer needle assembly 40 for cutting and sampling.

### 3.2 Delayed Fully Automatic Mode (Stylet Only)

Firstly, the above loading steps 1.1 and 1.2 are performed, then the biopsy needle 100 is controlled at a safe distance from a lesion, and then the above emission step 2.1 is performed to only emit the inner needle assembly 30. After emission of the inner needle assembly 30 is confirmed, the above emission step 2.2 is performed to emit the outer needle assembly 40 for cutting and sampling.

### 3.3 Fully Automatic Zero Range Mode

After the biopsy needle 100 is punctured into the lesion, the above loading step 1.1 is performed to load the outer needle assembly 40 in place, and then the above emission step 2.2 is performed to emit the outer needle assembly 40 for cutting and sampling.

In combination with the above usage modes, for easy to distinguish for users, as shown in FIG. 1, the first rear emission button 61 of the first rear emission assembly 60 and the first side emission button 81 of the first side emission assembly 80 are respectively marked with a letter “S”, corresponding to the delayed fully automatic mode mentioned above. The second rear emission button 71 of the second rear emission assembly 70 and the second side emission button 91 of the second side emission assembly 90 are respectively marked with a letter “A”, corresponding to the fully automatic emission mode mentioned above. Furthermore, as shown in FIG. 1 and FIG. 2, a label groove 192 for pasting a product label 193 is provided on the outer surface of the upper cover 10, and the product label 193 can be distinguished by different colors according to different needle models, making it convenient for users to choose and use.

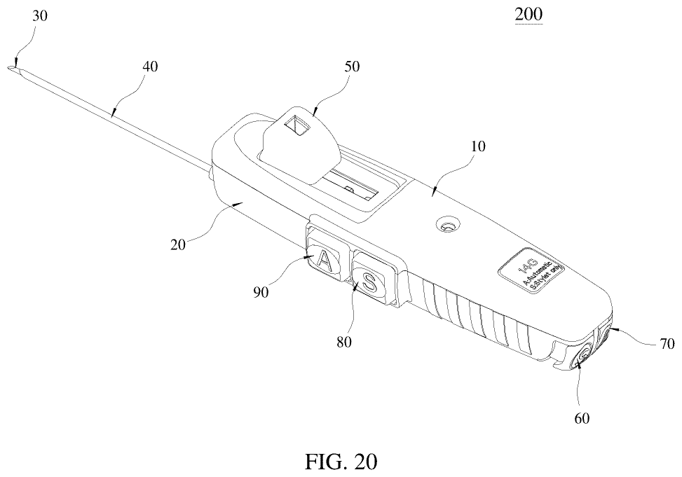

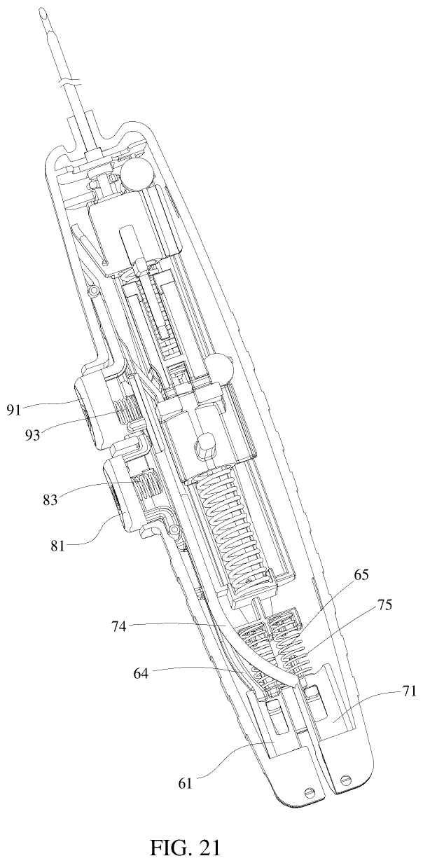

FIG. 20 and FIG. 21 illustrate schematic structural diagrams of a biopsy needle 200 with three usage modes according to a second embodiment of the present application. As shown in FIG. 20 and FIG. 21, the biopsy needle 200 according to the second embodiment of the present application has structures that are basically the same as the biopsy needle 100 according to the first embodiment mentioned above, with difference being that the first side emission assembly 80 and the second side emission assembly 90 are set separately from the first rear emission assembly 60 and the second rear emission assembly 70, respectively. The first side emission assembly 80 comprises a first side emission button 81 and a fourth reset spring 83. The first side emission button 81 is installed in a compressible manner in the installation position formed by a combination of the upper cover 10 and the base 20 and is reset by the fourth reset spring 83. Applying a force of pressing the first side emission button 81 inward can push corresponding emission structure on the first side emission button 81 inward against the first elastic sheet 323 that abuts to the first loading limiting surface 271, causing the first elastic sheet 323 to detach from the first loading limiting surface 271 and release the limitation, thereby the inner needle holder 32 and inner needle 31 are emitted under the action of the inner needle loading spring 33. After the force applied to the first side emission button 81 is released, the first side emission button 81 can be reset under the elastic force of the fourth reset spring 83. Similarly, the second side emission assembly 90 comprises a second side emission button 91 and a fifth reset spring 93. The second side emission button 91 is installed in a compressible manner in the installation position formed by a combination of the upper cover 10 and the base 20 and is reset by the fifth reset spring 93. Applying a force of pressing the second side emission button 91 inward can push corresponding emission structure on the second side emission button 91 inward against the second elastic sheet 423 that abuts to the second loading limiting surface 272, causing the second elastic sheet 423 to detach from the second loading limiting surface 272 and release the limitation, thereby the outer needle holder 42 and outer needle 41 are emitted under the elastic force of the outer needle loading spring 43. If the inner needle assembly 30 has not been emitted yet, pressing the second side emission button 91 inward can also push the first elastic sheet 323 that abuts to the first loading limiting surface 271 inward, causing the first elastic sheet 323 to detach from the first loading limiting surface 271 and release the limitation, thereby the inner needle holder 32 and the inner needle 31 are emitted under the action of the elastic force of the inner needle loading spring 33. After the force applied to the second side emission button 91 is released, the second side emission button 91 can be reset under the elastic force of the fifth reset spring 93.

The above is only the preferred embodiments of the present application and is not intended to limit the present application. Any modifications, equivalent substitutions and improvements made within the spirit and principles of the present application should be included within the protection scope of the present application.

I claim:

1. A biopsy needle, comprising an upper cover, a base, an inner needle assembly, and an outer needle assembly, wherein the inner needle assembly comprises an inner needle holder and an inner needle fixedly installed on the inner needle holder, the outer needle assembly comprises an outer needle holder and an outer needle fixedly installed on the outer needle holder, the inner needle holder and the outer needle holder are respectively installed in the upper cover and the base that are combined with each other in a reciprocating sliding and elastic loading manner in a needle insertion direction, and the inner needle and the outer needle extend from front end in the needle insertion direction of the upper cover and the base, and the inner needle is inserted into the outer needle, characterized in that the biopsy needle further comprises a loading assembly, a first rear emission assembly, a second rear emission assembly, a first side emission assembly and a second side emission assembly, wherein:
the loading assembly is slidably set on the upper cover back and forth in a direction parallel to the needle insertion direction, and a first loading matching structure is provided for the loading assembly and the outer needle holder respectively, by which the outer needle holder is driven to be elastically loaded in place when the loading assembly slides backward, and a second loading matching structure is provided for the loading assembly and the inner needle holder respectively, by which a cooperation is achieved after the outer needle holder is loaded in place to drive the inner needle holder to be elastically loaded in place with the loading assembly sliding backward;
the first rear emission assembly and the second rear emission assembly are respectively installed at rear end in the needle insertion direction in the upper cover and the base combined with each other, and the first rear emission assembly has a first rear emission button which acts on the inner needle holder to release a loading state of the inner needle holder and emit the inner needle holder, and the second rear emission assembly has a second rear emission button which acts on the outer needle holder to release a loading state of the outer needle holder and emit the outer needle holder, and acts on the inner needle holder to release the loading state of the inner needle holder and emit the inner needle holder when the inner needle holder has not been emitted; and
the first side emission assembly and the second side emission assembly are respectively installed on one side in the needle insertion direction in the upper cover and the base combined with each other, and the first side emission assembly has a first side emission button which acts on the inner needle holder to release the loading state of the inner needle holder and emit the inner needle holder, and the second side emission assembly has a second side emission button which acts on the outer needle holder to release the loading state of the outer needle holder and emit the outer needle holder, and acts on the inner needle holder to release the loading state of the inner needle holder and emit the inner needle holder when the inner needle holder has not been emitted.

2. The biopsy needle according to claim 1, characterized in that the loading assembly comprises a loading button, a loading seat, and a first reset spring, wherein the loading seat is slidably installed in a loading slot that extends parallel to the needle insertion direction on the upper cover, the loading button is fixedly connected to the loading seat and exposed outside the upper cover, and the first reset spring is set between the loading seat and the upper cover for resetting of the loading seat after it slides backward along the loading slot.

3. The biopsy needle according to claim 2, characterized in that the first loading matching structure comprises a loading rod extending downwards from a front end of the loading seat and a loading protrusion protruding on the outer needle holder, and the loading seat slides backwards along the loading slot to make the loading rod contact the loading protrusion, thereby driving the outer needle holder to slide backwards and be loaded in place.

4. The biopsy needle according to claim 3, characterized in that the second loading matching structure comprises two loading V-shaped grooves set on both sides of a rear end of the loading seat and two tongue pieces extending forward from the inner needle holder, and a connecting rod is connected between the two tongue pieces, and the outer needle holder further has an actuating rod that is inserted between the two tongue pieces of the inner needle holder to push the connecting rod upwards after the outer needle holder is loaded in place, to achieve a cooperation between the two tongue pieces and the two loading V-shaped grooves, and the loading seat slides backward along the loading slot to make the two loading V-shaped grooves contact against the two tongue pieces of the inner needle holder, thereby driving the inner needle holder to slide backward and be loaded in place.

5. The biopsy needle according to claim 1, characterized in that the inner needle holder has a first elastic sheet at its one side in the needle insertion direction, which extends diagonally forward relative to the needle insertion direction, and an inner needle loading spring is set between the inner needle holder and the base, and when the inner needle holder slides backwards driven by the loading assembly till a front end of the first elastic sheet abuts to a corresponding first loading limiting surface in the base, the inner needle holder is loaded in place; and the outer needle holder has a second elastic sheet at its corresponding side in the needle insertion direction, which extends diagonally forward relative to the needle insertion direction, an outer needle loading spring is set between the outer needle holder and the base, and when the outer needle holder slides backwards driven by the loading assembly till a front end of the second elastic sheet abuts to a corresponding second loading limiting surface in the base, the outer needle holder is loaded in place.

6. The biopsy needle according to claim 5, characterized in that both of the first rear emission button and the second rear emission button are installed on left and right sides at the rear end of the base in a compressible and elastic-reset manner, wherein the first rear emission button has a first rear emission member that extends towards the first elastic sheet of the inner needle holder, and when the first rear emission button is pressed, the first rear emission member pushes forward the first elastic sheet which abuts to the first loading limiting surface to make the first elastic sheet to detach from the first loading limiting surface and be released, thereby the inner needle holder and the inner needle are emitted under elastic force of the inner needle loading spring; and the second rear emission button has a second rear emission member that extends towards the second elastic sheet of the outer needle holder, and when the second rear emission button is pressed, the second rear emission member pushes forward the first elastic sheet of the inner needle holder that has not yet been emitted to make the first elastic sheet to detach from the first loading limiting surface and be released, thereby the inner needle holder and the inner needle are emitted under elastic force of the inner needle loading spring, and when the second rear emission button is continued to be pressed, the second rear emission member pushes forward the second elastic sheet of the outer needle holder that abuts to the second loading limiting surface to make the second elastic sheet to detach from the second loading limiting surface and be released, thereby the outer needle holder and the outer needle are emitted under elastic force of the outer needle loading spring.

7. The biopsy needle according to claim 6, characterized in that both of the first side emission button and the second side emission button are slidably installed back and forth in an installation position formed on one side of the upper cover and the base combined with each other, and the first side emission button is fixedly connected to the first rear emission member of the first rear emission button, and the second side emission button is fixedly connected to the second rear emission member of the second rear emission button.

8. The biopsy needle according to claim 6, characterized in that both of the first side emission button and the second side emission button are installed in a compressible and elastic-reset manner in an installation position formed on one side of the upper cover and the base combined with each other; and when the first side emission button is pressed, the first side emission button pushes inward the first elastic sheet which abuts to the first loading limiting surface to make the first elastic sheet to detach from the first loading limiting surface and be released, thereby the inner needle holder and the inner needle are emitted under elastic force of the inner needle loading spring; and when the second side emission button is pressed, the second side emission button pushes inward the first elastic sheet of the inner needle holder that has not yet been emitted to make the first elastic sheet to detach from the first loading limiting surface and be released, thereby the inner needle holder and the inner needle are emitted under elastic force of the inner needle loading spring, and when the second side emission button is continued to be pressed, the second side emission button pushes inward the second elastic sheet of the outer needle holder that abuts to the second loading limiting surface to make the second elastic sheet to detach from the second loading limiting surface and be released, thereby the outer needle holder and the outer needle are emitted under elastic force of the outer needle loading spring.

9. The biopsy needle according to claim 1, characterized in that the inner needle is fixedly set at a lower end of the inner needle holder, the outer needle is fixedly set at a lower end of the outer needle holder, and the inner needle holder and the outer needle holder are both limited from top, bottom, left and right in an inner space provided by the upper cover and the base combined with each other, to only move forward and backward in the needle insertion direction.

10. The biopsy needle according to claim 1, characterized in that a partition plate is set in the upper cover and the base combined with each other, for separating the first rear emission button and the second rear emission button.
